SEQUENCE LISTING (4 sequences)

SEQ ID NO: 1 - DNA, 19 bp, papillomavirus
gatccaacgtgtaagtctg

SEQ ID NO: 2 - DNA, 45 bp, papillomavirus
gggccatcagtcagcagtcgtagccggatccagacttacacgttg

SEQ ID NO: 3 - DNA, 22 bp, papillomavirus
ggccatcagtcagcagtcgtag

SEQ ID NO: 4 - DNA, 20 bp, papillomavirus
agggaatcccaatgaaggac

(19) United States
(12) Patent Application Publication
(10) Pub. No.: US 2010/0279416 A1
(43) Pub. Date: Nov. 4, 2010

(54) METHOD FOR INTRODUCING CHANGES INTO A EUKARYOTIC GENOME IN VIVO AND A KIT

(76) Inventors: Mart Ustav, Tartu (EE); Ene Ustav, Tartu (EE); Meelis Kadaja, Tartumaa (EE); Alina Sumerina, Tartu (EE)

Correspondence Address: REED SMITH LLP, P.O. BOX 488, PITTSBURGH, PA, 15230-0488, US

(21) Appl. No.: 12/593,558

(22) PCT Filed: Mar. 27, 2008
(86) PCT No.: PCT/EE08/00004

§ 371 Date: Jun. 17, 2010

(30) Foreign Application Priority Data
Mar. 28, 2007 (EE) P200700012

(51) Int. Cl. C12N 15/86; C12N 15/63

(52) U.S. Cl. 435/456; 435/320.1

(57) ABSTRACT

The invention relates to a method for introducing changes into a eukaryotic genome in vivo wherein the HPV genome, which comprises HPV replication origin sequence, is used together with HPV early proteins in order to achieve DNA replication in vivo. There is also disclosed a kit for in vivo amplification, excision, translocation and/or inversion of a DNA sequence, which comprises a vector carrying HPV genome or a part of HPV genome including HPV replication origin sequence, and expression vector or vectors encoding HPV early proteins.

### CROSS-REFERENCE TO RELATED APPLICATIONS

This application is a national phase application pursuant to 35 U.S.C. §371 of International Application No. PCT/EE2008/000004, filed Mar. 27, 2008, which claims priority to Estonia Application No. P200700012, filed Mar. 28, 2007.

### TECHNICAL FIELD OF THE INVENTION

The current invention is most widely in the field of molecular biology, more particularly in the field of virology and provides a means for introducing changes into a genome of a eukaryotic cell, which may be duplications, multiplications, insertions, deletions, inversions and/or translocations of a DNA sequence.

### BACKGROUND OF THE INVENTION

Papillomaviruses are small species-specific DNA tumor viruses that establish latent infection in the basal cells of the differentiating epithelium with the help of viral oncoproteins and maintain their small, about 8 kb, circular doublestranded (ds) genomes as episomal multicopy nuclear plasmids in proliferating transformed cells (Howley and Lowy, 2001) via action of the viral replication proteins E1 and E2 (Ustav and Stenlund, 1991; Chiang et al., 1992; Sverdrup and Khan, 1994). The E1 protein is the replication origin recognition factor and viral helicase (Yang et al., 1993; Sedman and Stenlund, 1998), which in cooperation with E2 facilitates recognition and effective loading of the host cell replication complexes at the papillomavirus origin in the upstream regulatory region (URR) (Ustav et al., 1991, 1993; Remm et al., 1992; Russell and Botchan, 1995; Stenlund, 2003).

In the latently infected basal cells, the high-risk (HR)-HPV genomes persist as episomal multicopy circular nuclear plasmids in order to support the viral life cycle. However, coexistence of the integrated and the episomal forms of HPV DNA in the same cells has been reported (Cooper et al., 1991; Kristiansen et al., 1994; Alazawi et al., 2002; Peitsaro et al., 2002a, b; Andersson et al., 2005; Arias-Pulido et al., 2006; Kulmala et al., 2006; Pett et al., 2006). The trigger for HPV integration is unclear, but linear fragments generated by dsDNA breaks of the genome or by ‘onion skin’ type of defective viral replication mode (Männik et al., 2002) may serve as substrates for integration (FIG. 7, steps 1 and 2). The ability of HPV-16 E7 to induce abnormal centrosome numbers is well known, which subsequently results in chromosome missegregation and aneuploidy (Duensing and Munger, 2002, 2004). Increase of E1 concentration above a certain level results in overly frequent loading of the viral hexameric helicase and cellular replication complexes at the viral origin and induction of ‘onion skin’-type replication intermediates on the viral genome (Männik et al., 2002). Consequently, complicated topological structures of the ‘onion skin’-type replication intermediates as well as heterogeneous linear subgenomic fragments are generated. Based on tissue tropism, more than 100 different types of human papillomaviruses (HPVs) are divided into subgroups of mucosal or cutaneous papillomaviruses; individual viruses from each group are designated ‘high risk’ or low risk' according to the propensity of the infected transformed cells for malignant progression (zur Hausen, 2002; de Villiers et al., 2004; Munger et al., 2004).

Cells carrying integrated HR-HPV sequences have selective growth advantage due to the increased cell immortalization (Romanczuk and Howley, 1992; Jeon et al., 1995). In the case of HPV18, HPV31 and HPV35, nearly 100% of the viral sequences show integration into the cancer cell genome. Integrated and episomal viral genomes are commonly found in the HPV16 DNA-positive cancers (Cooper et al., 1991; Kristiansen et al., 1994; Peitsaro et al., 2002a, b; Andersson et al., 2005; Arias-Pulido et al., 2006).

The papillomaviruses do not follow once-per-cell cycle replication mode (Ravnan et al., 1992; Piirsoo et al., 1996); therefore, multiple unscheduled initiation events at the functional integrated HPV origin could extend into the adjacent genomic locus and trigger rearrangements like deletions or duplications of the sequences of the cellular genome by repair/recombination machinery.

### DISCLOSURE OF THE INVENTION

The present invention is based on the ability of the HPV genome including its replication origin sequence to start DNA replication in two directions from the location of the replication origin sequence in the presence of HPV early proteins, in particular the E1 and E2 protein of various HPV strains. The authors have studied the events that may contribute to the formation of invasive cancers in the case of HR-HPV infections by demonstrating the effective HPV E1- and E2-dependent DNA amplification of the integrated HPV18 and HPV16 origins in HeLa and SiHa cells, respectively. The replication forks initiated at the integrated HPV origins extend into the flanking regions of cellular DNA. These amplified genomic sequences, resembling ‘onion skin’-type DNA replication intermediates as targets for the recombination and repair system that causes excisions of these sequences, resulting in de novo rearrangements and recombinations within the cellular DNA.

In result single-stranded or double-stranded DNA fragments were generated. These fragments possess biological activity. The fragments may be used as antisense DNA, DNAzyme or decoy-DNA. Moreover, any kind of these fragments may be used for therapeutical applications.

The integrated HR-HPV origin is effectively mobilized for replication by E1 and E2 produced from respective expression vectors, and from episomal HPV genomes transiently replicating in these cells. As a result, amplification of the integrated HR-HPV genome and flanking cellular DNA sequences occurs (FIG. 7, step 3), which can serve as targets for repair/recombination machinery. This results in rearrangements, including deletion as seen by the excision of HPV18 sequences or de novo integration of amplified sequences, as demonstrated by the analysis of the subclones (FIG. 7, step 4). One may assume that modifications of the genome providing gain of the function result in transformation of cells and will give a growth advantage to the cells after the loss of episomal viral genome.

Also de novo infection of papillomaviruses could result in intracellular mixture of episomal and integrated HPV and subsequent amplification of integrated HPV DNA together with flanking cellular sequences (FIG. 7, step 5). Therefore also the LR-HPVs is able to initiate DNA replication from the integrated HR-HPV origin. In addition to amplification of regulatory sequences or genes driving the cell cycle, there is also a possibility for the deletion of genomic sequences. The SiHa cell line itself is an example of such a process where cellular sequences flanking the HPV16 integration site (el Awady et al., 1987) lack approximately 300 kb within the intact chromosome 13 (according to the 36th assembly of human genome, NCBI, released in November 2005).

We can call it ‘hit-and-run’ mechanism as ‘lowrisk’ or ‘high-risk’ HPV episome itself could be lost quickly after the infection, but the damage caused by the chromosome-associated HR-HPV amplification remains.

The presented data suggest that papillomavirus DNA replication machinery can actively induce irreversible changes in the genomic make-up of the cell at sites of HPV origin integration. The results show that papillomavirus replication proteins are capable of mobilizing integrated HPV origin and that simultaneous DNA replication of episomal and integrated HPV origins may occur in HeLa cells. These kinds of changes provide a useful tool for research, if one wishes to amplify, excise or translocate a genomic sequence, either adjacent to the integration site of the HPV sequence or wishes to introduce foreign sequences to the cell with respectively constructed HPV vector. The kit including HPV replication origin and overexpression of genes encoding HPV E1 and E2 for introducing duplications, multiplications, insertions, deletions, inversions and/or translocations of a DNA sequence into a eukaryotic cell is particularly important as it exhibits unexpectedly good viability of the transfected cells thus making the system a good model for in vivo experimentation. Moreover, amplification of a DNA sequence encoding functional units of heredity allows this combination to serve as basis for gene expression and overexpression experiments. The beforementioned kit comprises a vector carrying HPV genome or a part of HPV genome including HPV replication origin sequence, and expression vector or vectors encoding HPV early proteins, e.g. E1 and E2.

### BRIEF DESCRIPTION OF THE DRAWINGS

FIG. 1 HPV E1 and E2 proteins initiate the replication from episomal and integrated HPV origins in HeLa cells.

(A, B) HeLa cells were cotransfected with plasmids for expression of homologous E2 and E1 from HPV6b (lanes 1-4), HPV11 (lanes 5-8), HPV16 (lanes 9-12) and HPV18 (lanes 13-16) together with 0.5 μg of pUCURR-6b, -11, -16 and -18, respectively. As controls, either 5 μg pMHE1-18 and 5 μg pQMNE2-18 (lanes 17 and 18) or 0.5 μg pUCURR18 alone (lanes 19 and 20) was transfected. A 3 μg measure of total DNA extracted 24 and 48 h after transfection was digested with HindIII/DpnI and analyzed by Southern blotting with radiolabeled pUC probe (A) or all four HPV URR sequences (B). (C) Schematic presentation of the HPV6b, -11, -16 and -18 E1 expression constructs. (D) Analysis of expression of HA epitopetagged E1 of HPV6b (lanes 1 and 2), HPV11 (lanes 3 and 4), HPV16 (lanes 5 and 6) and HPV18 (lanes 7 and 8) 48 h after transfection with 3F10-HRP antibody. (E) Schematic representation of the episomal HPV18 genome and the integrated HPV18 in HeLa cells (Lazo, 1987; Meissner, 1999). Cellular DNA is shown as dashed line and HPV18 DNA as solid line. Open arrows represent the viral ORFs and noncoding URR shown as an open box.

FIG. 2 Replication of integrated HPV18 is dependent on E1 protein concentration, while E2 has no effect.

(A, E) Increasing amounts of HPV18 E1 expression plasmids (from 0.5 to 10 μg) were cotransfected with 1 μg of HPV18 E2 expression vector. Total cellular DNA was extracted 24 h and 48 h after transfection and 3 μg of DNA was digested with HindIII (A) or BamHI (E) and analyzed by Southern blotting with ³²P-labeled HPV18 URR probe. (B) Southern blot analysis of HeLa cells transfected with HPV18 E1 vector (5 μg) and an increasing amount of HPV18 E2 vector (0.5-10 pg). DpnI was used to remove input plasmids. (C) Western blot analysis of HPV18 E1 protein expression in HeLa cells 48 h after transfection with 3F10-HRP antibody. (D) Western blot analysis of HPV18 E2 protein with 4E4 antibody. (E) Total cellular DNA was extracted at various time points from cells transfected with 1 μg pQME2-18 and 10 μg pMHE1-18 (lanes 1-6) or from mock-transfected cells (lanes 7-12); 3 pg from each DNA sample was digested with BamHI/DpnI and subjected to Southern blotting analysis with ³²P-labeled HPV18 URR-specific probe.

FIG. 3 Amplification of integrated HPV16 URR in SiHa cells transfected with HPV E1 and E2 expression vectors.

(A) Schematic presentation of the integrated HPV16 genome in chromosome 13 in SiHa cells. Viral ORFs are shown as open arrows and URR is indicated by an open box. Numbers in italics indicate the nucleotides of viral genome at the junction with cellular DNA. Acc65I restriction sites at nt 880 and nt 5378 of the viral genome are shown (B) SiHa cells were transfected. with expression vectors for E1 and E2 as follows: 2.5-20 μg of HPV6b E1 (lanes 1-4); 2.5-20 μg of HPV11 E1 (lanes 5-8); 1.2-5 μg of HPV16 E1 (lanes 9-11) and 1.2-5 μg of HPV18 E1 (lanes 10-12). A 5 μg measure of homologous E2 expression vectors was added for each transfection. For controls, either 5 μg pMHE1-16 alone (lane 13) or 5 μg pQMNE2-16 alone (lane 14) was transfected into the cells. The total DNA was isolated 24 h after transfection and 3 pg was digested with Acc65I/DpnI and analyzed by Southern blotting using radiolabeled HPV16 URR probe. (C) Analysis of E1 protein expression in SiHa cells 24 h after transfection using Western blot.

FIG. 4 Amplification of the integrated HPV16 origin and flanking cellular sequences induced by HR-HPV E1 and E2 proteins.

(A) Graphical representation of amplification of HPV16 URR and flanking sequences of two independent experiments, as described in (B). A 5 μg portion of pMHE1-16 and 5 μg pQMNE2-16 (dashed line in panel A and second row in panel B) or 5 μg pMHE1-18 and 5 μg pQMNE2-18 (solid line in panel A and third row in panel B) were transfected into SiHa cells. A 3 pg portion of total cellular DNA was extracted 24 h after transfection, digested with different enzyme combinations (indicated at the top in panel B, restriction sites shown in panel A) and then subjected to Southern blot analysis. Filters were probed with either radiolabeled HPV16 URR (lane 3 in panel B) or with cellular sequences (panel B, lanes 1, 2, 4 and 5) from various distances from both sides of the viral integration (SL1, SL2, SR1 and SR2). The replication signals were quantified on PhosphorImager and normalized to the signal from the mock-transfected SiHa cells. The average increase in copy numbers of different sequences is calculated (shown by vertical italic numbers in the graph in panel A). (C) Graphical representation of the results from two independent experiments, where 5 μg pQMNE2-18 together with increasing amounts of pMHE1-18 (2.5 μg, dotted line; 5 μg, dashed line; 10 μg, solid line) was transfected into SiHa cells. The replication signals were analyzed as described above and the average copy numbers corresponding to different sequences (URR-16, SL1, SL2, SR1, and SR2) are shown in the table (D).

FIG. 5 Coreplication of integrated and episomal HPV18 in HeLa cells.

(A) Low-molecular-weight DNA was extracted from HeLa cells at 48 and 96 h after transfection and (B) total DNA isolated from transfected HeLa cells at 48 h was analyzed by Southern blotting. A 3 μg portion of pUCHPV18 alone (lanes 2 and 3 in panel A and lane 1 in panel B), together with 10 μg pMHE1-18 (lanes 4 and 5 in panel A and lane 2 in panel B) or 10 μg pMHE1-18 and 2.5 μg pQMNE2-18 (lanes 6 and 7 in panel A and lane 3 in panel B), was transfected into HeLa cells. As controls, HeLa cells were transfected with 1 μg pUC-URR18 and 10 μg pMHE1-18 (lanes 8 and 9 in panel A and lane 4 in panel B), or together with 2.5 μg pQMNE2-18 (lanes 10 and 11 in panel A and lane 5 in panel B). A 5 μg portion of total DNA or half of the material from 60 mm dish (in case of low-molecular-weight DNA) was digested with HindIII/DpnI followed by Southern blot analysis with labeled HPV18 URR probe. Mock transfections (lane 1 in panel A and lane 6 in panel B) as well as 100 pg of HindIII-digested pUCHPV18 and pUC-URR18 are shown (lane 12 in panel A and lane 7 in panel B). (C) Southern blot analysis of the total population (lane 2) and sorted GFP-positive cells (lane 3) cotransfected with 10 pg pUCHPV18 plasmid and 0.5 μg pEGFP-N1. Sample from mock transfection is shown in lane 1. Markers of pUCHPV18 plasmid are shown in lanes 4-7; numbers indicated in the figure correspond to the copy number per cell. The total cellular DNA was extracted 48 h after transfection and 1.6 μg of it (corresponding to 2.5×10⁵ diploid cells) was analyzed in each lane as described above. The results from two independent experiments are presented in (D).

FIG. 6 Chromosomal rearrangements caused by the replication of integrated HPV16.

(A) Southern blot analysis of subclones from SiHa cells transfected with 10 μg pMHE1-16 alone (upper panel, lanes 1-43) or together with 5 μg pQMNE2-16 (lower panel, lanes 1-44). Single cell subcloning was performed 72 h after transfection. Total cellular DNA was extracted from each subclone and 3 μg of DNA was digested with BamHI and analyzed by Southern blot with radiolabeled HPV16 URR probe. Total DNA samples from SiHa cells were used for control (lane 44, upper panel; lane 45, lower panel). (B) HindIII, BglII and BamHI restriction analysis of subclones with rearrangements. Restriction pattern of control cells is shown in lanes 25-27. (C) Restriction map of SiHa chromosome 13 close to the HPV16 integration site. Lengths of the appropriate restriction fragments between two sites are depicted in parentheses. (D) Schematic representation of rearrangements at the HPV16 integration site in the third subclone as determined by DIPS-PCR. See Supplementary data for DNA sequence.

FIG. 7 Genomic instability of host cell induced by the HPV replication machinery.

Papillomavirus DNA is designated with −, host cell DNA is designated with = and different regions designated with -x-.

### DESCRIPTION OF EMBODIMENTS

### Example 1

### E1 Protein Induces DNA Replication in Cells with Integrated HPV

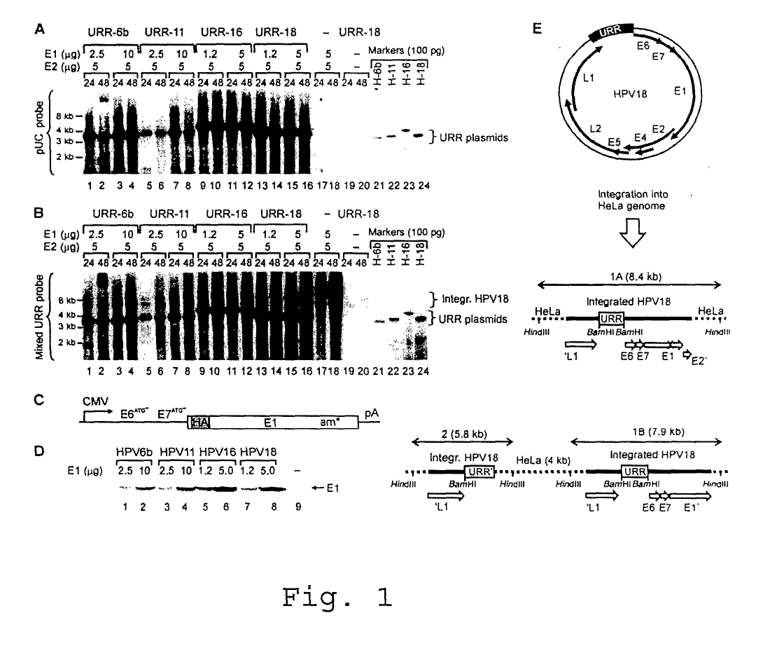

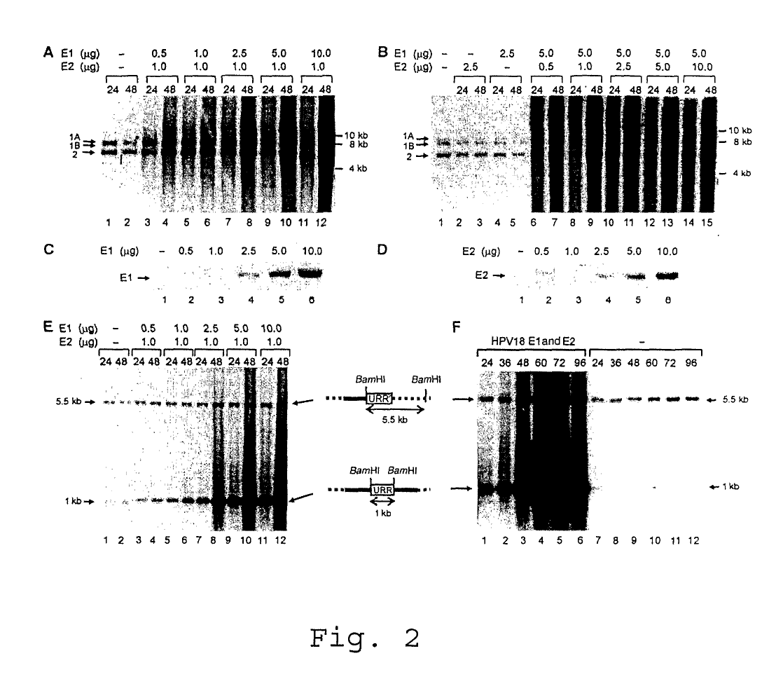

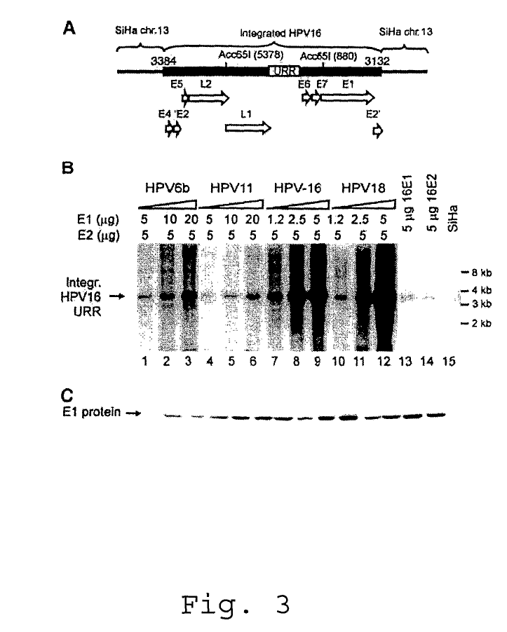

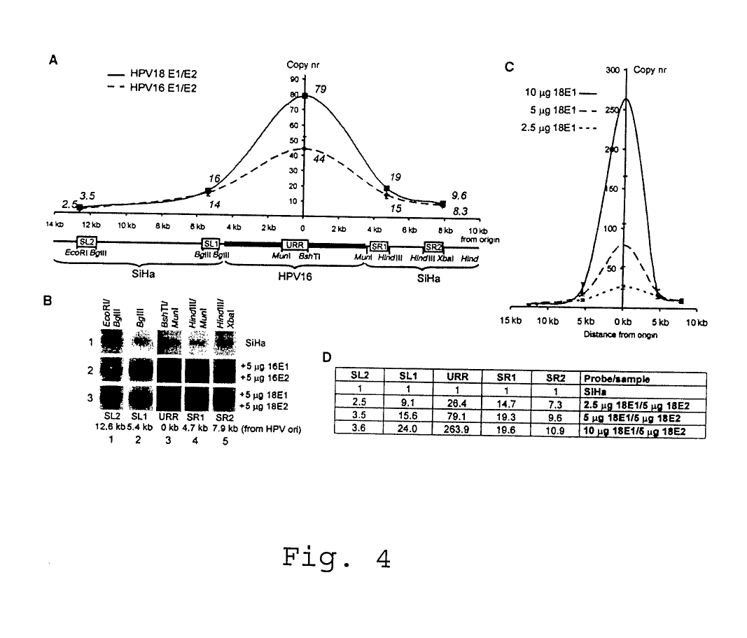

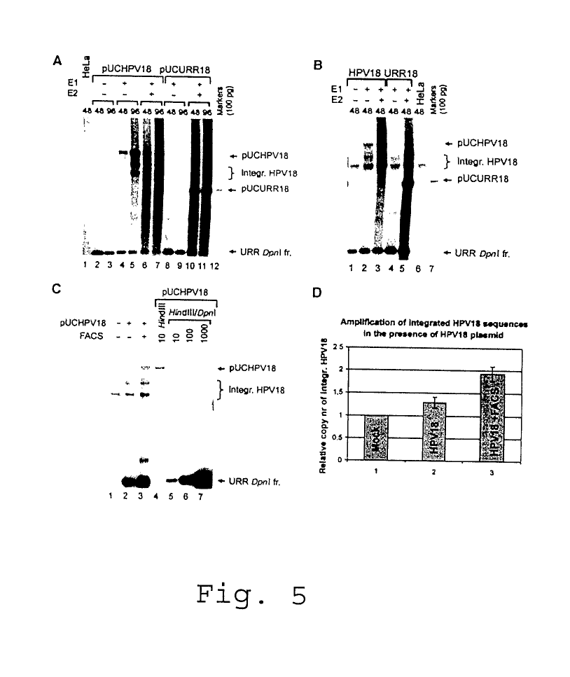

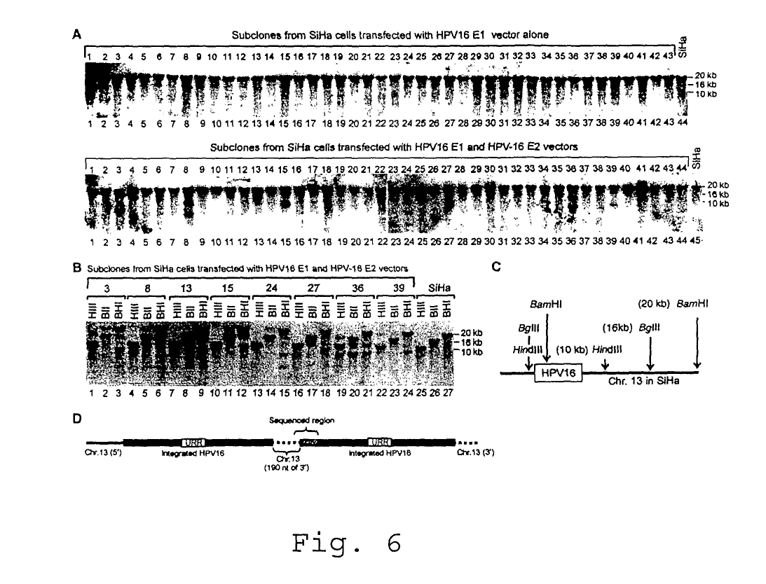

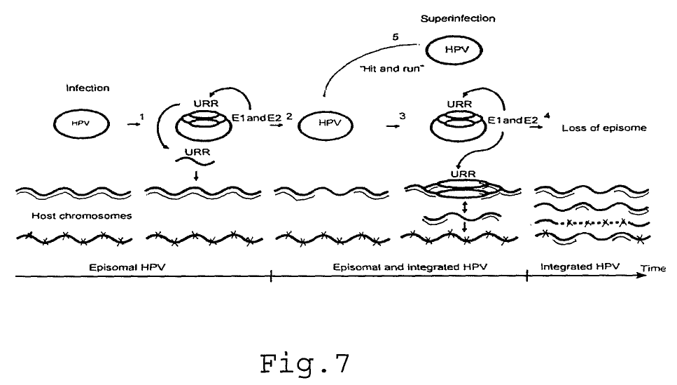

Titration of the HPV18 E1 and E2 proteins in the transient assays showed that the efficiency of DNA replication initiation depends on the E1 protein concentration (FIG. 2A), while modulation of E2 concentration in a quite wide range had little effect on the efficiency of initiation of the DNA replication of the integrated HPV (FIG. 2B). The replication reached a plateau at 0.5 pg of transfected E2 expression plasmid (FIG. 2B, lanes 6 and 7) and changed little at higher vector concentrations. The E2 expression level in our replication assays did not induce senescence or apoptosis of the transfected cells. High E1 levels caused smearing of the integrated HPV18 origin replication signals characteristic to the ‘onion skin’ type of replication mode. Cleavage of the integrated HPV18 sequences in HeLa cells (FIGS. 1A, B and E) with BamHI generates a 1 kb URR fragment, which includes the complete functional HPV replication origin and 5.5 kb URR fragment containing nonfunctional origin linked to cellular sequence. The kinetics of accumulation of amplification products with time was studied from 24 h to 96 h after transfection (FIGS. 2E and F). The amplification level of functional HPV18 URR was estimated up to 90 times at the 72 h time point compared with controls. Moderate amplification of a 5.5 kb fragment was detected at later time points. The amplification of 6-8 kb viral/cellular fragments in the presence of E1 and E2 (FIG. 2A) implies that DNA replication from HPV origin can extend into the flanking cellular sequences.

### Example 2

### Expression of E1 and E2 Proteins Induces Amplification of Episomal and Integrated HPV18 Origins in HeLa Cells

To test E1 expression constructs and to identify the conditions conducive to viral origin replication, the origin plasmids (pUC/URRs) of all HPVs, together with the homologous expression vectors for E1 and E2, were cotransfected into HeLa cells. The DpnI resistant replication signal from the transient assays was examined by Southern blot analysis of total DNA with common plasmid probe (FIG. 1A). Replication of all origin plasmids was clearly detected in HeLa cells. Western blot analysis to monitor E1 protein levels in transfected cells (FIG. 1D) confirms its effective and comparable expression in HeLa cells. E2 levels were kept constant and considerably low (5 pg of transfected E2 plasmid) in order to support replication without suppression of E6 promoter. HPV18 and HPV18 E1 proteins seem to be the most efficient in initiation of DNA replication within the used expression range, while HPV6B and HPV11 E1 proteins are clearly less efficient. In addition to the unit-sized plasmid, a smear of newly synthesized products with considerably larger and smaller size than input was detected, especially in the case of HR-HPV replication proteins (FIG. 1A, lanes 9-12 and 13-16). Probing of the same blots with URR-specific probes showed additional replication signals of different intensity in every lane across the panel (FIG. 1B, compare with panel A). These signals most probably result from the multiple integrated HPV18 URRs located in at least five different sites of the host genome (Lazo, 1987; Meissner, 1999). It has been demonstrated that also the URR with origin is present in addition to the coding sequences for viral oncoproteins E6 and E7 (schematically shown in FIG. 1E). According to Lazo and Meissner, the HindIII digest generates three fragments of 8.4 kb (1A), 7.9 kb (1B) and 5.8 kb, carrying HPV18 sequences (FIG. 1E). The URR-specific probe for HPV18 detected amplification of exactly these fragments (FIG. 1B). The intensities of these bands correlated with the E1 expression levels and were the strongest in the lanes of HR-HPV E1 expression constructs (FIG. 1B, lanes 9-18). LR-HPV E1 proteins were less efficient for replicating integrated HPV18 origin, which may be explained by lower specificity toward the HPV18 origin, although expression level of the E1 proteins was comparable. Intensive replication signal was detected also in the control lanes, where only the viral expression vectors were transfected, suggesting that integrated HPV18 origin is fully competent for initiation of DNA replication (FIG. 1B, lanes 17 and 18). These results show that papillomavirus replication proteins are capable of mobilizing integrated HPV origin and that simultaneous DNA replication of episomal and integrated HPV origins may occur in HeLa cells.

### Example 3

### HPV E1 and E2 Proteins Efficiently Initiate Replication from Integrated HPV16 Origin in SiHa Cells

SiHa cell line derived from the cervical carcinoma of a 55-year-old Japanese female is aneusomic and has been found to contain 66-72 chromosomes. However, this cell line has been shown to be disomic with respect to chromosome 13, which contains one copy of the HPV16 genome (Meissner, 1999; Szuhai et al., 2000). Integration of the HPV16 genome has occurred, with disruption in the E2 and E4 ORFs at nucleotides 3132 and 3384 of HPV16 genome (FIG. 3A). Expression plasmids for the E1 and E2 proteins were transfected into SiHa cells and replication of integrated HPV16 origin DNA was analyzed using HPV16 URR probe. The E2 expression plasmid concentration was kept constant at the level where replication of integrated HPV16 had reached a plateau (data not shown). Increasing amounts of E1 expression plasmids were used in replication assays. In order to reach comparable levels of E1 expression, much higher amounts of HPV6b and HPV11 E1 expression plasmids had to be used in the transfection mixture (FIGS. 3B and C). The results were interpreted using a previously derived physical map of the integration site of HPV16 (Meissner, 1999), shown schematically in FIG. 3A. Untransfected SiHa cells as well as cells transfected with HPV16 E1 or E2 plasmids alone gave the faint, similar-strength signal of 3.4 kb after the digestion of an equal amount of total DNA samples with Acc65I (FIG. 3B, lanes 13-15). Cotransfection of the E2 plasmid with increasing amounts of the E1 vector resulted in a significant increase in the HPV16 URR-specific signal in the case of all four HPVs. This indicates that no endogenous expression of functional E1 or E2 protein could be detected in these cells. Expression vectors for the HPV16 (lanes 7-9) and HPV18 E1 proteins (lanes 10-12) induced most efficient initiation of DNA replication of the integrated HPV16 origin when compared with LR-HPV (lanes 1-3) and (lanes 4-6). Remarkably, at higher concentrations of E1 protein of HR-HPVs (lanes 8 and 9, 11 and 12), a heterogeneous mixture of fragments that did not migrate in the gel as a specific band was generated. Analysis of the replication products by 2D gel indicated formation of ‘onion skin’-type replication intermediates and linear dsDNA fragments as shown for BPV1 (Männik et al., 2002) and HPV16 in SiHa (data not shown). This confirms that integrated replication origins of HR-HPV types are functionally active for the HPV E1- and E2-dependent initiation of replication.

### Example 4

### Estimation of the Size of the Integrated HPV Replicon

The replication competence of the integrated HPV origins directed by viral replication proteins brings up an intriguing possibility that flanking cellular sequences on both sides of viral integration could be coamplified. We determined the size of the replicon also in SiHa cells by measuring the amplification levels of cellular sequences at various distances from integrated viral DNA replication origin. The HPV16 genome is integrated at chromosome 13q21-31 in SiHa cells. The integrated HPV16 as well as the flanking cellular DNA have been sequenced (Baker et al., 1987; Meissner, 1999). The 50 and 30 flanking cellular DNA sequences, determined by Baker et al., were subjected to BLAST search against the NCBI 36 assembly of human genome (released in November 2005). According to the results, 50 viral-cellular junction in SiHa was located at 72 686 871 by and 30 viral-cellular junction at 72 984 815 by of chromosome 13. This suggests that the distance between these junctional sequences is normally approximately 300 kb, indicating that considerable loss of genomic DNA has taken place in the process of integration of HPV16 DNA. Coordinates of the viral-cellular junctions, determined by BLASTsearch, were used to identify and amplify cellular sequences at various distances from integrated HPV16 using PCR. Sequence blocks with the furthermost nucleotide at 5.4 and 12.6 kb upstream (SL1, SL2 in FIG. 4A) and 4.7 and 7.9 kb downstream of HPV16 on (SR1, SR2 in FIG. 4A), together with URR sequence itself, were used as probes in the following quantitative Southern blot analysis. The goal was to determine the amplification level of genomic sequences at different distances on both sides of the integrated HPV origin in SiHa cells, cotransfected with expression plasmids for E1 and E2 proteins of either HPV16 (FIG. 4B, row 2) or HPV18 (FIG. 4B, row 3). The control transfection was performed with the carrier DNA alone (FIG. 4B, row 1). The cellular DNA from transfected cells was digested with appropriate combinations of enzymes (lanes 1-5 in FIG. 4B; location of restriction sites is depicted in the drawing in FIG. 4A) and three analogous blots (with SiHa DNA, +HPV16 E1 and E2, and +HPV18 E1 and E2) were probed with four appropriate radiolabeled cellular sequences or URR probe (shown at the bottom of lanes 1-5 in FIG. 4B). The hybridization signals of two independent experiments were quantified by Phosphorimager and normalized to the carrier-transfected SiHa cells (FIG. 4A). The data show that DNA replication, initiated from the HPV16 origin, will extend into the flanking cellular sequences on both sides and that the DNA replication fork travels a distance of at least 12.6 kb from the HPV16 origin, on average, as analyzed at 24 h after transfection. That makes the total size of the amplicon to be more than 25 kb. We estimated that, under the experimental conditions used, the transfection efficiency was approximately 40%. This means that more than half of the signal comes from the cells that do not have E1- and E2-induced amplification. This suggests that at the cellular level amplification as well as the replicon size is likely to be more than the estimated. values. The replication proteins of HPV18 were more efficient in initiating DNA replication of the HPV16 URR compared with HPV16 proteins (solid line compared with dashed line in graph in FIG. 4A). However, there are almost no differences in the amplification of distal cellular sequences (SL1, SL2, SR1 and SR2) between these sets of replication proteins. Furthermore, we observed that even if the replication of integrated HPV16 origin is highly dependent on E1 concentration (FIGS. 4C and D), the E1 concentration dependence of amplification of distal sequences decreases with the distance from the replication origin. There was a 10 times difference in the amplification signal of the HPV16 URR when 2.5 or 10 μg of HPV18 E1 plasmid was used, but less than 1.5 times difference in amplifying cellular sequences that are about 10 kb away (SL2 and SR2). These data suggest that overexpression of E1 protein may generate replication intermediates, locked for elongation, owing to the topological constraints in the ‘onion skin’ structures that would prevent traveling of replication forks further into the genome. Therefore, elongation of replication forks at considerable distances from the initiation site is determined presumably more by proper configuration of the template and replication complex functioning at it and less by the E1 level. These data also suggest that low E1 and E2 protein levels, for example from episomal HPV genome, and respective extension of the E1-driven replication into the flanking sequences may be highly relevant for induction of amplification of the HPV integration locus in HPV-infected cells.

### Example 5

### Coreplication of Integrated and Episomal HPV18 in HeLa Cells

HeLa cells were transfected with the plasmid carrying functional full-size HPV18 genome cloned into pUC19 plasmid. Extrachromosomal supercoiled plasmids from the transfected cells were extracted at certain time points by alkaline lysis. Analysis of the episomal replication products (provided in FIG. 5A) shows that DpnI-resistant and HindIII-linearized HPV replication induced genomic instability HPV18 viral genome replication could be detected in HeLa cells at low level (FIG. 5A, lanes 2 and 3), indicating that viral genome is basically functional and directs the expression of E1 and E2 replication proteins in some cells, although at low level. Cotransfection of HPV18 plasmid with E2 expression vector did not increase replication signal (data not shown); however, cotransfection of E1 expression vector with HPV18 genome considerably increased the replication signal of episomal HPV18 genome as well as showed a clear increase in the signal from integrated HPV18 sequences, especially at later time points (FIG. 5A, lanes 4 and 5). This suggests that expression of E2 protein from the viral genome is sufficient to support E1-driven initiation of DNA replication of episomal as well as integrated HPV18 replication origins in HeLa cells. Detection of a clear, strong and increasing-in-time replication signal of the integrated HPV18 HindIII fragments in the extrachromosomal fraction suggests that excision of replicating integrated HPV18 sequences from the chromosomal DNA occurs, which is further followed by extrachromosomal replication of these plasmids. Cotransfection of HPV18 genome with E1 and E2 expression vectors tremendously increased HPV18 DNA replication (FIG. 5, lanes 6 and 7). The smear detected in the Southern blots is presumably caused by single-stranded DNA fragments extracted from the ‘onion skin’ replication intermediates during alkaline extraction. The control experiment, where pUCURR18 plasmid was used, showed replication signal only in the presence of E1 and E2 proteins (FIG. 5, lanes 8 and 9 compared with 10 and 11), as expected. Analysis of total genomic DNA from the same transfection after 48 h is presented in FIG. 5B. A relatively smaller fraction of cells was analyzed in this blot and it shows that without isolation of episomal DNA, the replication of HPV18 DNA is detected only in the case of cotransfection with E1 and E2 expression vectors (FIG. 5B, lanes 2, 3 and 5). In order to evaluate the ability of the HPV18 genome to mobilize the integrated replication origin, we cotransfected cloned HPV18 genome together with EGFP expression vector (pEGFP-N1) into HeLa cells. The EGFP-positive cells were isolated using cell sorter FACS-ARIA 48 h after transfection. This allowed us to analyze only the transfected cells and remove background of integrated HPV sequences of untransfected cells. Total cellular DNA was digested with HindIII/DpnI and analyzed by Southern blotting. First, we showed an increase in the average apparent copy number of input episomal HPV18 plasmid by four-fold, from 40 to 170 per cell, as estimated by the intensity of the URR containing DpnI fragment (FIG. 5C). We reproducibly succeeded in detecting the replication of episomal HPV18 plasmid at the level of eight copies per cell. Most importantly, we also detected amplification of integrated HPV18 sequences by two-fold in these cells (FIG. 5C, lane 3 and D, third bar). These data suggest that E1 and E2 proteins, expressed from the native context of the HPV18 genome in HeLa cells, initiate episomal DNA replication and also endogenous levels of these proteins produced from the episomal genome are sufficient for mobilization of the integrated HPV origin locus for unscheduled replication. These data provide experimental proof for the hypothesis that similar amplification of the integrated HRHPV sequences could occur in HPV-infected cells as in LG-SIL and HG-SIL cells if episomal HPV genomes producing replication proteins are present.

### Example 6

### Replication from Integrated HPV16 Origin Will Cause Chromosomal Rearrangements in SiHa Cells

Amplified HPV locus in SiHa cells is the potential target for repair/recombination machinery that may result in chromosomal alterations. The cells transfected with low amounts of HPV16 E1 and E2 expression plasmids were single cell subcloned 72 h after transfection. The subclones were expanded, total DNA was extracted and purified, and the HPV16 integration patterns of subclones were analyzed by Southern blot analysis using URR probe. Alterations in the restriction pattern of the integrated HPV16 locus could be detected only in cases when at least one of the cleavage sites for specific restriction enzyme is outside of the DNA fragment that was involved in rearrangement. Therefore, BamHI cleavage, which generates 21.5 kb DNA fragment from 1.8 kb upstream to B20 kb downstream of HPV16 URR, was used (FIG. 6C). Analysis of 43 tested subclones, generated after transfection with the E1 expression plasmid alone, did not reveal any changes in the BamHI restriction pattern (FIG. 6A, upper panel). However, eight out of 44 subclones isolated from E1- and E2-transfected cells contained a novel HPV16 restriction pattern (lower panel in FIG. 6A, lanes 3, 8, 13, 15, 24, 27, 36 and 39), representing either an internal rearrangement or reintegration at a novel site. Additional analysis of these subclones with HindIII and BglII (sites in the scheme of FIG. 6C) confirmed the rearrangement of the HPV16 URR containing sequences and showed that every subclone resulted from the individual event of repair/recombination (FIG. 6B). Thus, cells can survive the considerable changes induced by the HPV DNA replication machinery, which results in effective rearrangement of the genomic DNA. More precise analysis of one of the subclones (FIG. 6B, lanes 1-3) by DIPS-KR suggests that in situ duplication of HPV16 genome together with 30 cellular DNA (FIG. 6D) has occurred. We determined that the nucleotide 190 of chromosome 13 from the 30 end of initially integrated HPV16 has been linked to nucleotide 3852 (within the E2 stop codon) of the HPV16 genome. Sequence homology could not be found between these sites, indicating that illegitimate recombination has taken place.

It is believed that the methods and examples shown or described above have been characterized as preferred, various changes and modifications may be made therein without departing from the scope of the invention as defined in the following claims.

### Materials and Methods

Plasmids

Plasmids pQMNE2-6b, -11, -16 and -18 were prepared by cloning E2 ORFs from HPV6b (2723-3829 nt), -11 (2723-3826 nt), -16 (2756-3853 nt) and -18 (2817-3914 nt) into the multicloning site of eukaryotic expression vector pQM-NTag/Ai+(Quattromed Ltd) followed by deletion of intron. The pUCURR-6b, -11, -16 and -18 plasmids were cloned by inserting viral sequences containing the URR region of HPV6b (7292-101 nt), -11 (7022-94 nt), -16 (6361-282 nt) and -18 (6929-124 nt) into the multicloning site of pUC-19 plasmid. pMHE1-6b, -11, -16 and -18 vectors contained E6, E7 and E1 ORFs from HPV6b (102-2781 nt), -11 (102-2781 nt), -16 (83-2814 nt) or -18 (105-2887 nt) that were directed by CMV promoter in the pQM-NTag/Ai+vector with deleted intron and 3F12 epitope tag. Initiation codons for E6 and E7 oncogenes were deleted. The major splice donor site (AGGT) at the beginning of E1 ORFs was disrupted by inserting influenza hemagglutinin epitope tag (HA) inframe into the E1 coding sequence. The inserted HA tag had no effect on the E1 protein activities. Additional point mutation was introduced into the splicing acceptor site of HPV11 E1 ORF (2622 nt), (ACA-ACC), and this did not change the coding capacity. pUCHPV18 wt were cloned by inserting EcoRV-linearized HPV-18 genome (4670 nt within L2) into pUC19 SmaI site.

Cell Lines and Transfection

HeLa and SiHa cells were grown in Iscove's modified Dulbecco's medium (IMDM) supplemented with 10% fetal calf serum. Electroporation experiments were carried out as described by Ustav and Stenlund, 1991, using the Bio-Rad Gene Pulser II apparatus supplied with a capacitance extender (Bio-Rad Laboratories, USA). Capacitance was set to 975 mF and voltage to 220V in all experiments. Cells were plated onto 60 mm dishes and harvested at different time points.

Transient Replication Assays

Low-molecular-weight DNA was purified by alkaline lysis as described by Ustav and Stenlund, 1991. Total DNA was extracted from cells (FM Ausubel et al., 1998). DNA digested with appropriate enzymes was resolved in 0.5 or 0.8% agarose gel, blotted and hybridized with appropriate 32P-labeled probe generated by random priming (DecaLabel kit, Fermentas, Lithuania). The cellular sequences (SL1, SR2, SL1 and SL2) used in hybridization were amplified from SiHa genomic DNA with PCR using Taq polymerase and primers that were designed with the programs Primer3 (Rozen and Skaletsky, 2000) and GenomeTester (Andreson et al., 2006). Radioactive signals were quantified using ImageQuant software of PhosporImager SI (Molecular Dynamics, Amersham Biosciences, UK).

Immunoblotting

Total protein from an equal number of cells was separated by electrophoresis on 10% polyacrylamide-SDS gels and transferred to Immobilon-P membrane (Millipore, USA). Antibodies 3F10-HRP (Roche) and 4E4 were used to detect E1 and E2 proteins using the enhanced chemoluminescence detection kit (Amersham Biosciences).

Cell Sorting

For cell sorting, cells were cotransfected with pEGFP-N1 (Clonetech) and pUCHPV18 wt plasmids. Forty-eight hours after transfection, the transfected cells were sorted on the basis of EGFP fluorescence using the FACSDiva software and the FACSAria hardware (Becton Dickinson) equipped with a 13 mW argon ion laser set at 488 nm with a 530/30 nm filter. The purity of EGFP+cells, when reanalyzed, was 90±5%.

DIPS-PCR

DIPS-PCR assay was performed as described previously (Luft et al., 2001). The ds adapter was constituted from AS1 (5′-PO₄-gatccaacgtgtaagtctg-NH₂) (SEQ. ID NO. 1) and AL1 (5′-gggccatcagtcagcagtcgtagccggatccagacttacacgttg-3′) (SEQ. ID NO. 2) DNA oligos. The primers used in PCR were AP1 (5′-ggccatcagtcagcagtcgtag-3′) (SEQ. ID NO. 3) and S1 (5′. agggaatcccaatgaaggac-3′) (SEQ. ID NO. 4). PCR products were analyzed by 1.2% agarose gel electrophoresis followed by purification of the product of interest and sequence determination.

1-21. (canceled)

22. A method for introducing changes into a eukaryotic genome located in a eukaryotic cell, comprising the steps of:
incorporating an HPV genome into said eukaryotic genome, wherein said HPV genome includes an HPV replication origin sequence;
introducing HPV early proteins into said eukaryotic cell;
enabling the replication process in said eukaryotic cell; and
reintegrating the changes of the eukaryotic genome into the native eukaryotic sequences.

23. A method for overexpressing genomic DNA sequences in a eukaryotic cell, comprising the steps of:
incorporating an HPV genome into said genomic DNA, wherein said HPV genome includes an HPV replication origin sequence;
introducing HPV early proteins into said eukaryotic cell;
enabling proceeding of replication of DNA in said eukaryotic cell; and
enabling expression from said nucleic acid sequences multiplied due to an interactive effect on replication of the presence of HPV replication origin sequence and HPV early proteins.

24. A method according to claim 1, wherein said HPV replication origin sequence together with said HPV early proteins are used for introducing mutations into the genome of a eukaryotic cell.

25. A method according to claim 3, wherein the introduced mutations are duplications, multiplications, insertions, deletions, inversions and/or translocations of said genomic DNA sequence.

26. A method according to claim 1, wherein said HPV replication origin sequence together with HPV early proteins are used for excision of site-specific genomic DNA sequences within said genomic DNA sequence.

27. A method according to claim 1, wherein said HPV replication origin sequence together with HPV early proteins are used for insertion of site-specific inserted DNA sequences within said genomic DNA sequence.

28. A method according to claim 1, wherein said HPV replication origin sequence together with HPV early proteins are used for amplification of said genomic DNA sequences.

29. A method according to claim 1, wherein said HPV replication origin sequence together with HPV early proteins are used for amplification of site-specific inserted DNA sequences.

30. A method according to claim 1, wherein said HPV replication origin sequence together with HPV early proteins are used for translocating genomic sequences.

31. A method according to claim 1, wherein said HPV early proteins comprise HPV E1 and E2 proteins.

32. A method according to claim 1, wherein said HPV replication origin sequence is co-expressed with genes encoding HPV early protein E1 and HPV early protein E2.

33. A method according to claim 11, further comprising the step of simultaneous overexpression of the genes encoding HPV E1 and E2 proteins.

34. A method according to claim 12, comprising the step of integrating said HPV replication origin sequence and said genes encoding HPV E1 and E2 proteins into the host cell genome.

35. A method according to claim 12, wherein the HPV sequences are episomal.

36. A method according to claim 1, wherein said genomic DNA sequences into which changes have been introduced are amplified.

37. A method according to claim 15, wherein the amplified DNA includes single-stranded or double stranded DNA fragments.

38. A method according to claim 16, wherein the amplified DNA fragment possesses biological activity.

39. A method according to claim 17, wherein the amplified DNA fragment is antisense-DNA, DNAzyme, or decoy-DNA.

40. A method according to claim 16, wherein the amplification of the DNA fragment is used for therapeutic purposes.

41. A kit for in vivo amplification, excision, translocation and/or inversion of a DNA sequence, which comprises a vector carrying HPV genome or a part of HPV genome including HPV replication origin sequence, and expression vector or vectors encoding HPV early proteins.

42. A kit according to claim 20, wherein said HPV early proteins are E1 and E2 proteins.
